(19) United States
(12) Patent Application Publication
(10) Pub. No.: US 2004/0043489 A1
(43) Pub. Date: Mar. 4, 2004

(54) Gene delivery vectors provided with a tissue tropism for dendritic cells and methods of use

(76) Inventors: Menzo Havenga, Alphen a/d Rijn (NL); Ronald Vogels, Linschoten (NL); Abraham Bout, Moerkapolle (NL)

Correspondence Address: TRASK BRITT, P.O. BOX 2550, SALT LAKE CITY, UT, 84110, US

(21) Appl. No.: 10/646,449

(22) Filed: Aug. 22, 2003

Related U.S. Application Data
(62) Division of application No. 09/665,472, filed on Sep. 20, 2000.
Application No. 09/665,472 is a continuation-in-part of application No. 09/348,354, filed on Jul. 7, 1999.

(30) Foreign Application Priority Data
Jul. 8, 1998 (EP) EP 98202297.2

(51) Int. Cl. C12N 15/861

(52) U.S. Cl. 435/456

(57) ABSTRACT

Adenoviral vectors can be used in vaccines to cause antigen-presenting cells to display desired antigens. Disclosed is a vector and associated methods that transduce antigen-presenting cells better than currently available vectors, enabling the vector to be delivered in lower doses, and thus improves the efficiency of adenoviral vaccine technology.

### RELATED APPLICATIONS

This application is a division of U.S. application Ser. No. 09/665,472, filed Sep. 20, 2000, which is a continuation-in-part of U.S. application Ser. No. 09/348,354, filed Jul. 7, 1999, which claims the benefit of European application EP 98202297.2, filed Jul. 8, 1998.

### TECHNICAL FIELD

The present invention relates generally to the field of biotechnology, particularly to gene delivery vehicles having a tissue tropism for dendritic cells, the tissue tropism for dendritic cells being provided by a viral capsid protein.

### BACKGROUND

In gene therapy, genetic information is usually delivered to a host cell in order to either correct (supplement) a genetic deficiency in the cell, to inhibit an undesired function in the cell, or to eliminate the host cell altogether. Of course, the genetic information can also be intended to provide the host cell with a desired function, for instance, to supply a secreted protein to treat other cells of the host, etc.

Many different methods have been developed to introduce new genetic information into cells. Although many different systems may work on cell lines cultured in vitro, only the group of viral vector mediated gene delivery methods seems to be able to meet the required efficiency of gene transfer in vivo. Thus, for the purposes of gene therapy, most attention has been directed toward the development of suitable viral vectors, such as vectors based on adenovirus.

Such adenoviral vectors can deliver foreign genetic information very efficiently to target cells in vivo. Moreover, obtaining large amounts of adenovirus vectors are, for most types of adenovirus vectors, not a problem. Adenovirus vectors are relatively easy to concentrate and purify. Moreover, clinical studies have provided valuable information on the use of these vectors in patients.

Many reasons exist for using adenovirus vectors to deliver nucleic acid to target cells in gene therapy protocols. However, some characteristics of the current vectors limit their use in specific applications. For instance, endothelial cells and smooth muscle cells are not easily transduced by the current generation of adenoviral vectors. For many gene therapy applications, these types of cells should be genetically modified. In some applications, however, even the very good in vivo delivery capacity of adenovirus vectors is insufficient, and higher transfer efficiencies are required. This is the case, for instance, when most cells of a target tissue need to be transduced.

Adenoviruses contain a linear double-stranded DNA molecule of approximately 36,000 base pairs (“bp”). This molecule contains identical inverted terminal repeats (“ITRs”) of approximately 90-140 base pairs with the exact length depending on the adenovirus serotype. The viral origins of replication are within the ITRs at the genome ends. The transcription units are divided into early and late regions.

Shortly after infection, the E1A and E1B proteins are expressed and function in transactivation of cellular and adenoviral genes. The early regions E2A and E2B encode proteins (DNA binding protein, pre-terminal protein and polymerase) required for the replication of the adenoviral genome (reviewed in van der Vliet, 1995). The early region E4 encodes several proteins with pleiotropic functions, for example, transactivation of the E2 early promoter, facilitating transport and accumulation of viral mRNAs in the late phase of infection and increasing nuclear stability of major late pre-mRNAs (reviewed in Leppard, 1997). The early region 3 encodes proteins that are involved in modulation of the immune response of the host (Wold et al., 1995). The late region is transcribed from one single promoter (major late promoter) and is activated at the onset of DNA replication. Complex splicing and polyadenylation mechanisms give rise to more than 12 RNA species coding for core proteins, capsid proteins (penton, hexon, fiber and associated proteins), viral protease and proteins necessary for the assembly of the capsid and shut-down of host protein translation (Imperiale et al. “Post-transcriptional Control of Adenovirus Gene Expression”, The Molecular Repertoire of Adenoviruses I., pp. 139-171. (W. Doerfler and P. Böhm (editors), Springer-Verlag Berlin Heidelberg 1995).

The interaction of the virus with the host cell has mainly been investigated with the serotype C viruses Ad2 and Ad5. Binding occurs via interaction of the knob region of the protruding fiber with a cellular receptor. The receptor for Ad2 and Ad5 and probably more adenoviruses is known as the “Coxsackievirus and Adenovirus Receptor” or “CAR” protein (Bergelson et al., 1997). Internalization is mediated through interaction of the RGD sequence present in the penton base with cellular integrins (Wickham et al., 1993). This may not be true for all serotypes, for example, serotypes 40 and 41 do not contain a RGD sequence in their penton base sequence (Kidd et al., 1993).

The initial step for successful infection is binding of adenovirus to its target cell, a process mediated through fiber protein. The fiber protein has a trimeric structure (Stouten et al, 1992) with different lengths depending on the virus serotype (Signas et al., 1985; Kidd et al., 1993). Different serotypes have polypeptides with structurally similar N and C termini, but different middle stem regions. The first 30 amino acids at the N terminus are involved in anchoring of the fiber to the penton base (Chroboczek et al., 1995), especially the conserved FNPVYP region in the tail (Arnberg et al, 1997). The C-terminus, or “knob”, is responsible for initial interaction with the cellular adenovirus receptor. After this initial binding, secondary binding between the capsid penton base and cell-surface integrins leads to internalization of viral particles in coated pits and endocytosis (Morgan et al., 1969; Svensson and Persson, 1984; Varga et al., 1991; Greber et al., 1993; Wickham et al, 1993). Integrins are αβ-heterodimers of which at least 14 α-subunits and 8 β-subunits have been identified (Hynes, 1992). The array of integrins expressed in cells is complex and will vary between cell types and cellular environment. Although the knob contains some conserved regions, between serotypes, knob proteins show a high degree of variability, indicating that different adenovirus receptors exist.

At present, six different subgroups of human adenoviruses have been proposed which in total encompass approximately 50 distinct adenovirus serotypes. Besides these human adenoviruses, many animal adenoviruses have been identified (see, e.g., Ishibashi and Yasue, 1984). A serotype is defined on the basis of its immunological distinctiveness as determined by quantitative neutralization with animal antiserum (horse, rabbit). If neutralization shows a certain degree of cross-reaction between two viruses, distinctiveness of serotype is assumed if A) the hemagglutinins are unrelated, as shown by lack of cross-reaction on hemagglutination-inhibition, or B) substantial biophysical/biochemical differences in DNA exist (Francki et al., 1991). The serotypes identified last (42-49) were isolated for the first time from HIV infected patients (Hierholzer et al., 1988; Schnurr et al., 1993). For reasons not well understood, most of such immuno-compromised patients shed adenoviruses that were never isolated from immuno-competent individuals (Hierholzer et al., 1988, 1992; Khoo et al., 1995).

Besides differences towards the sensitivity against neutralizing antibodies of different adenovirus serotypes, adenoviruses in subgroup C such as Ad2 and Ad5 bind to different receptors as compared to adenoviruses from subgroup B such as Ad3, Ad7, Ad 11, Ad 14, Ad21, Ad34, and Ad35 (see, e.g., Defer et al., 1990; Gall et al., 1996). Likewise, it has been demonstrated that receptor specificity could be altered by exchanging the Ad3 knob protein with the Ad5 knob protein, and vice versa (Krasnykh et al., 1996; Stevenson et al., 1995, 1997). Serotypes 2, 4, 5 and 7 all have a natural affiliation towards lung epithelia and other respiratory tissues. In contrast, serotypes 40 and 41 have a natural affinity for the gastrointestinal tract. These serotypes differ in at least capsid proteins (penton-base, hexon), proteins responsible for cell binding (fiber protein), and proteins involved in adenovirus replication. It is unknown to what extent the capsid proteins determine the differences in tropism found between the serotypes. It may very well be that post-infection mechanisms determine cell type specificity of adenoviruses. It has been shown that adenoviruses from serotypes A (Ad12 and Ad31), C (Ad2 and Ad5), D (Ad9 and Ad15), E (Ad4) and F (Ad41) all are able to bind labeled, soluble CAR (sCAR) protein when immobilized on nitrocellulose. Furthermore, binding of these adenovirus serotypes to Ramos cells, that express high levels of CAR but lack integrins (Roelvink et al., 1996), could be efficiently blocked by addition of sCAR to viruses prior to infection (Roelvink et al., 1998). However, the fact that (at least some) members of these subgroups are able to bind CAR does not exclude that these viruses have different infection efficiencies in various cell types. For example, subgroup D serotypes have relatively short fiber shafts compared to subgroup A and C viruses. It has been postulated that the tropism of subgroup D viruses is to a large extent determined by the penton base binding to integrins (Roelvink et al., 1996; Roelvink et al., 1998). Another example is provided by Zabner et al., 1998 who tested 14 different serotypes on infection of human ciliated airway epithelia (“CAB”) and found that serotype 17 (subgroup D) was bound and internalized more efficiently then all other viruses, including other members of subgroup D. Similar experiments using serotypes from subgroup A-F in primary fetal rat cells showed that adenoviruses from subgroup A and B were inefficient whereas viruses from subgroup D were most efficient (Law et al., 1998). Also in this case viruses within one subgroup displayed different efficiencies. The importance of fiber binding for the improved infection of Ad17 in CAE was shown by Armentano et al. (International Patent Appln. WO 98/22609) who made a recombinant LacZ Ad2 virus with a fiber gene from Ad17 and showed that the chimeric virus infected CAE more efficient then LacZ Ad2 viruses with Ad2 fibers.

Thus, despite their shared ability to bind CAR differences in the length of the fiber, knob sequence and other capsid proteins, for example, penton base of the different serotypes may determine the efficiency by which an adenovirus infects a certain target cell. Of interest in this respect is the ability of Ad5 and Ad2 fibers (but not of Ad3 fibers) to bind to fibronectin III and MHC class 1 α2 derived peptides. This suggests that adenoviruses are able to use cellular receptors other than CAR (Hong et al., 1997). Serotypes 40 and 41 (subgroup F) are known to carry two fiber proteins differing in the length of the shaft. The long shafted 41L fiber is shown to bind CAR whereas the short shafted 41S is not capable of binding CAR (Roelvink et al., 1998). The receptor for the short fiber is not known.

Most adenoviral gene delivery vectors currently used in gene therapy are derived from the serotype C adenoviruses Ad2 or Ad5. The vectors have a deletion in the E1 region, where novel genetic information can be introduced. The E1 deletion renders the recombinant virus replication defective. It has been demonstrated extensively that recombinant adenovirus, in particular serotype 5 is suitable for efficient transfer of genes in vivo to the liver, the airway epithelium and solid tumors in animal models and human xenografts in imuuno-deficient mice (Bout 1996, 1997; Blaese et al., 1995).

Gene transfer vectors derived from adenoviruses (adenoviral vectors) have a number of features that make them particularly useful for gene transfer:

1) the biology of the adenoviruses is well characterized,

2) the adenovirus is not associated with severe human pathology,

3) the virus is extremely efficient in introducing its DNA into the host cell,

4) the virus can infect a wide variety of cells and has a broad host-range,

5) the virus can be produced at high titers in large quantities, and

6) the virus can be rendered replication defective by deletion of the early-region 1 (E1) of the viral genome (Brody and Crystal, 1994).

However, a number of drawbacks are still associated with the use of adenoviral vectors:

1) Adenoviruses, especially the well investigated serotypes Ad2 and Ad5, usually elicit an immune response by the host into which they are introduced,

2) it is currently not feasible to target the virus to certain cells and tissues,

3) the replication and other functions of the adenovirus are not always very well suited for the cells, which are to be provided with the additional genetic material, and

4) the serotypes Ad2 or Ad5, are not ideally suited for delivering additional genetic material to organs other than the liver.

The liver can be particularly well transduced with vectors derived from Ad2 or Ad5. Delivery of such vectors via the bloodstream leads to a significant delivery of the vectors to the cells of the liver. In therapies where other cell types than liver cells need to be transduced, some means of liver exclusion must be applied to prevent uptake of the vector by these cells. Current methods rely on the physical separation of the vector from the liver cells, most of these methods rely on localizing the vector and/or the target organ via surgery, balloon angioplasty or direct injection into an organ via for instance needles. Liver exclusion is also being practiced through delivery of the vector to compartments in the body that are essentially isolated from the bloodstream thereby preventing transport of the vector to the liver. Although these methods mostly succeed in avoiding gross delivery of the vector to the liver, most of the methods are crude and still have considerable leakage and/or have poor target tissue penetration characteristics. In some cases, inadvertent delivery of the vector to liver cells can be toxic to the patient. For instance, delivery of a herpes simplex virus (“HSV”) thymidine kinase (“TK”) gene for the subsequent killing of dividing cancer cells through administration of gancyclovir is quite dangerous when also a significant amount of liver cells are transduced by the vector. Significant delivery and subsequent expression of the HSV-TK gene to liver cells is associated with severe toxicity.

Dendritic cells are antigen presenting cells (“APC”), specialized to initiate a primary immune response. They are also able to boost a memory type of immune response. Dependent on their stage of development, dendritic cells display different functions: immature dendritic cells are very efficient in the uptake and processing of antigens for presentation by Major Histocompatibility Complex (“MHC”) class I and class II molecules, whereas mature dendritic cells, being less effective in antigen capture and processing, perform much better at stimulating naive and memory CD4⁺ and CD8⁺ T cells, due to the high expression of MHC molecules and co-stimulatory molecules at their cell surface. The immature DCs mature in vivo after uptake of antigen, travel to the T-cell areas in the lymphoid organs, and prime T-cell activation.

Since DCs are the cells responsible for triggering an immune response, there has been a long standing interest in loading DCs with immunostimulatory proteins, peptides, or the genes encoding these proteins, to trigger the immune system. The applications for this strategy are in the field of cancer treatment as well as in the field of vaccination. So far, anti-cancer strategies have focused primarily on ex vivo loading of DCs with antigen (protein or peptide). These studies have revealed that this procedure resulted in induction of cytotoxic T cell activity. The antigens used to load the cells are generally identified as being tumor specific. Some, non-limiting, examples of such antigens are GP 100, mage, or Mart-I for melanoma.

Besides treating cancer, many other potential human diseases are currently being prevented through vaccination. Well-known examples of disease prevention via vaccination strategies include hepatitis A, B, and C, influenza, rabies, yellow fever, and measles. Besides these well-known vaccination programs, research programs for treatment of malaria, ebola, river blindness, HIV and many other diseases are being developed.

Many of the identified pathogens are considered too dangerous for the generation of “crippled” pathogen vaccines. It would thus be an improvement in the art to be able to isolate and characterize proteins of each pathogen to which a “full blown” immune response is mounted, thus resulting in complete protection upon challenge with wild type pathogen.

### BRIEF SUMMARY OF THE INVENTION

In the herein described vaccination strategy, a “crippled” pathogen is presented to the immune system via the action of the antigen presenting cells, i.e., immature DCs.

According to the invention, adenoviral vectors are used in vaccines to cause antigen-presenting cells to display desired antigens. Disclosed are vectors and associated means and methods which transduce antigen-presenting cells better than currently available vectors, enabling the vector to be delivered in lower doses thus improving the efficiency of the adenoviral vaccine technology.

As is more thoroughly described herein, such a gene delivery vehicle is provided with at least a tissue tropism for dendritic cells. In such a gene delivery vehicle, the tissue tropism for dendritic cells is generally provided by a virus capsid. The virus capsid preferably includes protein fragments derived from at least two different viruses, such as an adenovirus (e.g., an adenovirus of subgroup B, such as a fiber protein derived from a subgroup B adenovirus). An adenovirus capsid with (or provided with) a tissue tropism for dendritic cells may have the capsid comprising proteins from at least two different adenoviruses and at least a tissue tropism determining fragment of a fiber protein is derived from a subgroup B adenovirus.

The cell line PER.C6™ (IntroGene, by (now Crucell Holland by, Leiden, N L) can be used to produce vaccines by producing adenoviral vectors that can safely deliver a portion of a pathogen's DNA into the body, provoking an immune response against the disease.

The invention also includes pharmaceutical compositions, such as vaccines, that include the gene delivery vehicle of the invention and use of the composition to treat or prevent disease.

### BRIEF DESCRIPTION OF THE DRAWINGS

In the drawings, which illustrate what is currently considered to be the best mode for carrying out the invention:

FIG. 1: Transduction of immature dendritic cells (“DCs”) at a virus dose of 100 or 1000 virus particles per cell. Virus tested is Ad5 and Ad5 based vectors carrying the fiber of serotype 12 (Ad5.Fib12), 16 (Ad5.Fib16), 28 (Ad5.Fib28), 32 (Ad5.Fib32), the long fiber of 40 (Ad5.Fib40-L, 49 (Ad5.Fib49), 51 (Ad5.Fib51). Luciferase transgene expression is expressed as relative light units per microgram of protein.

FIG. 2: Flow cytometric analyses of LacZ expression on immature and mature DCs transduced with 10000 virus particles per cell of Ad5 or the fiber chimeric vectors Ad5.Fib16, Ad5.Fib40-L, or Ad5.Fib51. Percentages of cells scored positive are shown in the upper right corner of each histogram.

FIG. 3: Luciferase transgene expression in human immature DCs measured 48 hours after transduction with 1000 or 5000 virus particles per cell. Viruses tested were fiber chimeric viruses carrying the fiber of subgroup B members (serotypes 11, 16, 35, and 51).

FIG. 4: GFP expression in immature human DCs48 hours after transduction with 1000 virus particles per cell of Ad5, Ad5.Fib16, and Ad5.Fib35. Non-transduced cells were used to set a background level of approximately 1% (−).

FIG. 5: Transduction of mouse and chimpanzee DCs. Luciferase transgene expression measured in mouse DCs 48 hours after transduction is expressed as relative light units per microgram of protein. Chimpanzee DCs were measured 48 hours after transduction using a flow cytometer. GFP expression demonstrates the poor transduction of Ad (35) in contrast to Ad5.Fib35 (66%).

FIG. 6 is a graph charting relative light units (“RLU”) per 10⁴ DC for various recombinant fiber modified vectors.

FIG. 7 consists of two graphs charting GFP expression determined 24 hours after virus exposure. The results are expressed as (a) percentage GFP positive cells and (b) median fluorescence intensity. Dosages used: 103, 104, or 10⁵ virus particles per DC (white bar, grey bar, or black bar respectively).

FIG. 8 consists of two graphs (a and b) comparing (a) percentage GFP positive cells and (b) median fluorescence intensity after immature DCs were treated with LPS to allow maturation of the DC. Matured DCs were incubated with Ad5.GFP or Ad5Fib16.GFP, Ad5Fib35.GFP, Ad5fib40-L.GFP, or Ad4.Fib51.GFP. Dosages used were: 10³ 10⁴, or 10⁵ virus particles per DC (white bar, grey bar, or black bar respectively).

FIG. 9 is a bar graph showing % GFP+DC (percentage GFP positive cells) for various recombinant fiber-modified vectors. The maturation agents used were LPS (black bars), TNF-a (white with black dots), MCM (diagonal downward), poly I:C (black with white dots), and anti-CD40 antibodies (diagonal upwards). As a negative control for a maturation marker, IFN-a (grey bars) was used. Immature DC s were used a control (white bars).

FIG. 10 consists of three bar graphs (a, b, and c). The DC types were immature DC (white bar), mature DC (black bar), or immature DC (grey bar) transduced and subsequently matured using LPS. Shown are (a) percentage GFP positive cells detected, (b) median fluorescence intensity, and (c) cells that were frozen and the genomic DNA extracted to quantify the number of adenoviral genomes using real-time PCR.

FIG. 11 consists of three graphs comparing IFN-gamma production by immature DCs transduced with 10⁵, 10⁴ or 10³ virus particles (top, middle, and bottom, respectively) of Ad5.gp100 or Ad5.Fib35.gp100 (white squares and circles, respectively). Likewise, matured DC were transduced with 10⁵, 10⁴ or 10³ virus particles of Ad5.gp100 or Ad5.Fib35.gp100 are depicted (black squares and circles, respectively).

### DETAILED DESCRIPTION OF THE INVENTION

A gene delivery vehicle according to the invention preferably has at least one of the protein fragments comprising a tissue tropism determining fragment of a fiber protein derived from a subgroup B adenovirus. Preferably, at least one of the protein fragments comprises a tissue tropism determining fragment of a fiber protein derived from a subgroup B adenovirus. A still more preferred gene delivery vehicle has at least one of the protein fragments comprising a tissue tropism determining fragment of a fiber protein derived from a subgroup B adenovirus, such as adenovirus 16.

In one embodiment, however, a gene delivery vehicle according to the invention further includes protein fragments derived from an adenovirus of subgroup C.

Also, the gene delivery vehicle can include a nucleic acid derived from one or more adenovirus.

In one embodiment, the gene delivery vehicle according to the invention has a nucleic acid comprising at least one sequence encoding a fiber protein comprising at least a tissue tropism determining fragment of a subgroup B adenovirus fiber protein, preferably of adenovirus 16.

Furthermore, the adenovirus nucleic acid can be modified such that the capacity of the adenoviral nucleic acid to replicate in a target cell has been reduced or disabled or the adenoviral nucleic acid can be modified so that the capacity of a host immune system to mount an immune response against adenoviral proteins encoded by the adenovirus nucleic acid has been reduced or disabled.

A gene delivery vehicle according to the invention can comprise a minimal adenovirus vector or an Ad/AAV chimeric vector and can comprise at least one non-adenovirus nucleic acid.

To load DCs with immunostimulatory proteins or peptides to become therapeutically feasible at least two distinct criteria have to be met. First, the isolation of large numbers of DCs that can be isolated, manipulated, and re-infused into a patient, making the procedure autologous. To date, it is possible to obtain such large quantities of immature DCs from cultured peripheral blood monocytes from any given donor. Second, a vector that can transduce DCs efficiently such that the DNA encoding for an immunostimulatory protein can be delivered. The latter is extremely important since it has become clear that the time required for DCs to travel to the lymphoid organs is such that most proteins or peptides are already released from the DCs, resulting in incomplete immune priming. Because DCs are terminally differentiated and thus non-dividing cells, recombinant adenoviral vectors are being considered for delivering the DNA encoding for antigens to DCs. Ideally, this adenovirus should have a high affinity for dendritic cells, but should also not be recognized by neutralizing antibodies of the host such that in vivo transduction of DCs can be accomplished. The latter would obviate the need for ex vivo manipulations of DCs but would result in a medical procedure identical to the vaccination programs that are currently in place, i.e., intramuscular or subcutaneous injection predominantly. Thus, dendritic cells transduced by adenoviral vectors encoding an immunogenic protein may be ideally suited to serve as natural adjuvants for immunotherapy and vaccination.

Efficient gene delivery to DCs is a major interest in the field of gene therapy. Therefore, alteration of the Ad5 host cell range to be able to target DCs in vitro as well as in vivo is a major interest of the invention. To identify a chimeric adenovirus with preferred infection characteristics for human DCs, we generated a library of Ad5 based viruses carrying the fiber molecule from alternative serotypes (serotypes 8, 9, 13, 16, 17, 32, 35, 45, 40-L, 51). Ad5 was included as a reference.

As more thoroughly herein, the susceptibility of human monocyte derived immature and mature dendritic cells to recombinant chimeric adenoviruses expressing different fibers was evaluated.

The invention is further explained by the use of the following illustrative examples:

### EXAMPLES

### Example I

An Ad5/fiber35 chimeric vector with cell type specificity for dendritic cells.

Human PBMC from healthy donors were isolated through Ficoll-Hypaque density centrifugation. Monocytes were isolated from PBMC by enrichment for CD14⁺ cells using staining with FITC labeled anti-human CD 14 monoclonal antibody (Becton Dickinson), anti-FITC microbeads, and MACS separation columns (Miltenyi Biotec).

This procedure usually results in a population of cells that are <90% CD14+as analyzed by FACS. Cells were placed in culture using RPMI-1640 medium (Gibco) containing 10% Foetal Bovine Serum (“FBS”) (Gibco), 200 ng/ml rhu GM-CSF (R&D/ITK diagnostics, 100 ng/ml rhu IL-4 (R&D/ITK diagnostics) and cultured for 7 days with feeding of the cultures with fresh medium containing cytokines on alternate days. After 7 days, the immature dendritic cells resulting from this procedure express a phenotype CD83⁻,CD14^{low} or CD14⁻,HLA-DR⁺, as was demonstrated by FACS analysis.

Immature DCs were matured by culturing the cells in a medium containing 100 ng/ml TNF-a for 3 days, after which, they expressed CD83 on their cell surface.

### Example II

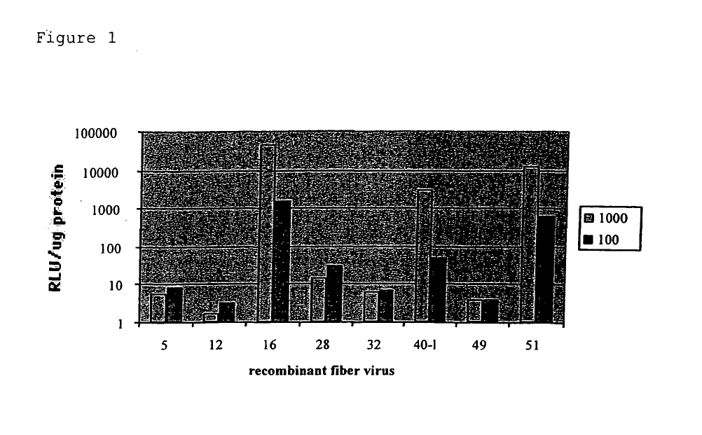

5×10⁵ immature DCs were seeded in wells of 24-well plates and exposed for 24 hours to 100 and 1000 virus particles per cell of each fiber recombinant virus. Virus tested was Ad5, and the fiber chimeric viruses based on Ad5: Ad5.Fib12, Ad5.Fib16, Ad5.Fib28, Ad5.Fib32, Ad5.Fib40-L (long fiber of serotype 40), Ad5.Fib49, and Ad5.Fib51 (where Fibxx stands for the serotype from which the fiber molecule is derived). These viruses are derived from subgroup C, A, B, D, D, F, D, and B respectively. After 24-hours, cells were lysed (1% Triton X-100/PBS) and luciferase activity was determined using a protocol supplied by the manufacturer (Promega, Madison, Wis., USA). The results of this example, shown in FIG. 1, demonstrate that Ad5 poorly infects immature DCs as evidenced by the low level of transgene expression. In contrast, Ad5.Fib16 and Ad5.Fib51 (both a B-group fiber chimeric virus) and also Ad5.Fib40-L (Subgroup F) show efficient infection of immature DCs based on luciferase transgene expression.

### Example III

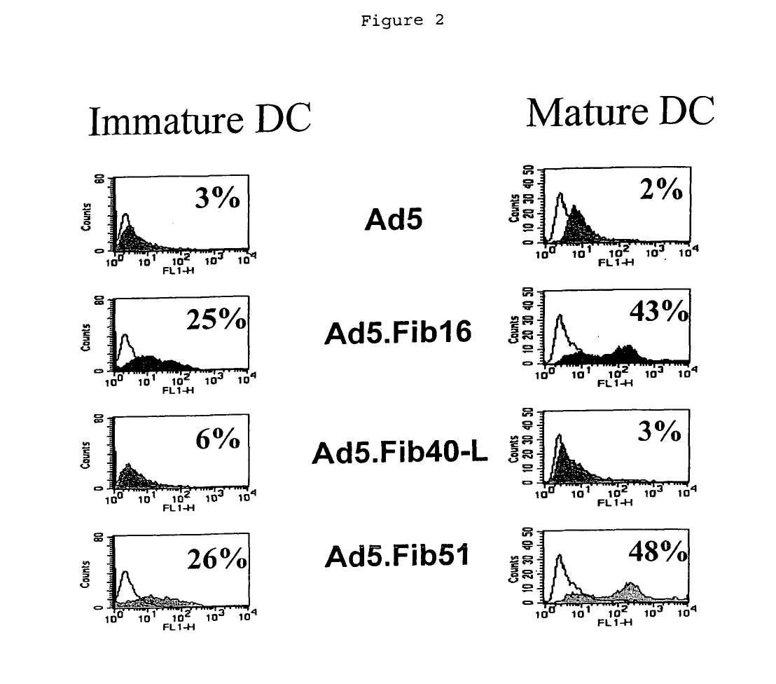

In a second experiment, 5×10⁵ immature and mature dendritic cells were infected with 10,000 virus particles per cell of Ad5, Ad5.Fib16, Ad5.Fib40-L, and Ad5.Fib51 all carrying the LacZ gene as a marker. LacZ expression was monitored by flow cytometric analysis using a CM-FDG kit system and the instructions supplied by the manufacturer (Molecular Probes, Leiden, NL). The results of this experiment, shown in FIG. 2, correlate with the previous experiment in that Ad5.Fib16 and Ad5.Fib51 are superior to Ad5 in transducing mature and immature human DCs. Also, this example shows that Ad5.Fib40-L is not as good as Ad5.Fib16 and Ad5.Fib51, but is better than Ad5.

### Example IV

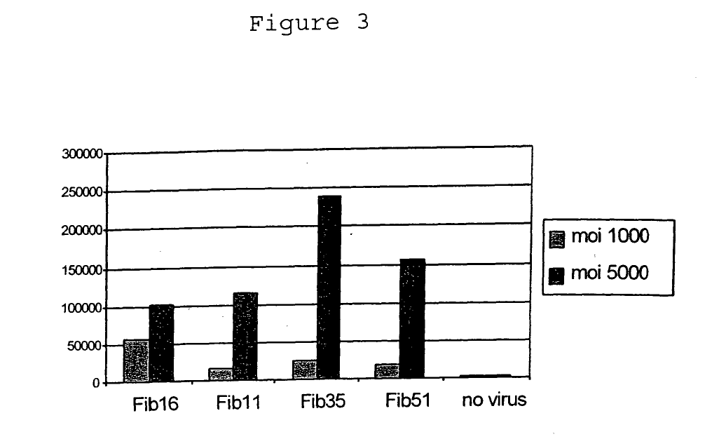

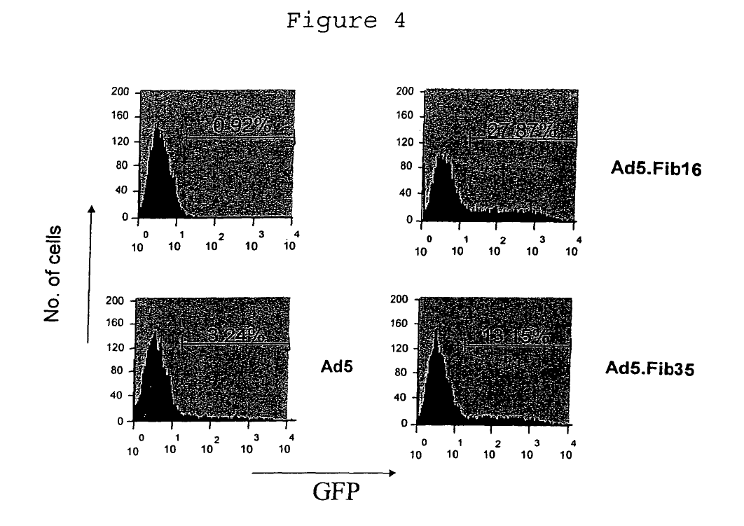

Based on the earlier Examples, we tested other chimeric adenoviruses containing fibers of B group viruses, for example, Ad5.Fib11 and Ad5.Fib35 for their capacity to infect DCs. We focused on immature DCs, since these are the cells that process an expressed transgene product into MHC class I and II presentable peptides. Immature DC's were seeded at a cell density of 5×10⁵ cells/well in 24 well plates (Costar) and infected with 1,000 and 5,000 virus particles per cell after which the cells were cultured for 48 hours under conditions for immature DCs prior to cell lysis and Luciferase activity measurements. The results of this Example, shown in FIG. 3, demonstrate that Ad5 based chimeric adenoviruses containing fibers of group-B viruses efficiently infect immature DCs. In a fourth experiment, we again infected immature DCs identically as described in the former experiments but this time Ad5, Ad5.Fib16, and Ad5.Fib35 were used carrying GFP as a marker gene. The results on GFP expression measured with a flow cytometer 48 hours after virus exposure are shown in FIG. 4, and correlate with the data obtained so far. Thus, the results so far are consistent in that Ad5 based vectors carrying a fiber from an alternative adenovirus derived from subgroup B predominantly fiber of 35, 51, 16, and 11 are superior to Ad5 for transducing human DCs.

### Example V

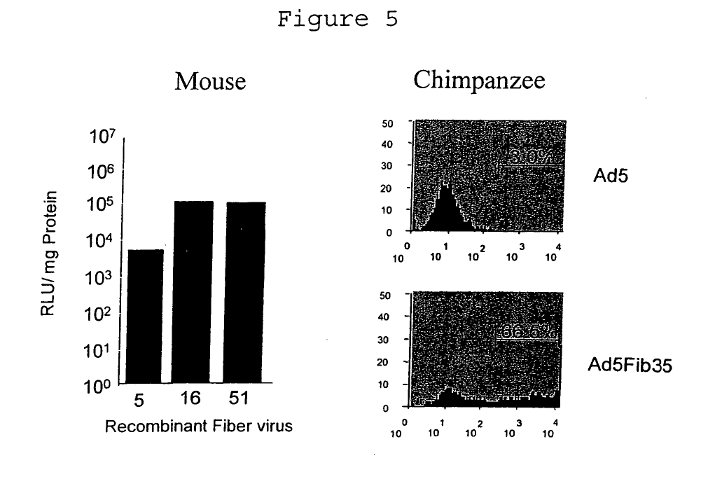

The adenoviruses disclosed herein are also very suitable for vaccinating animals. To illustrate this, we tested DCs derived from mice and chimpanzees to identify whether these viruses could be used in these animal models. Chimpanzees in particular, since the receptor for human adenovirus derived from subgroup B is unknown to date and therefore it is unknown whether this protein is conserved among species. For both species, immature DCs were seeded at a density of 10⁵ cells per well of 24-well plates. Cells were subsequently exposed for 48 hours to 1000 virus particles per cell of Ad5, Ad5Fib16, and Ad5.Fib51 in case of mouse dendritic cells and Ad5, and Ad.Fib35 in case of chimpanzee DCs (see, FIG. 5). The mouse experiment was performed with viruses carrying luciferase as a marker, and demonstrated approximately 10-50 fold increased luciferase activity as compared to Ad5.

The chimpanzee DCs were infected with the GFP viruses, and were analyzed using a flow cytometer. These results (also shown in FIG. 5) demonstrate that Ad5 (3%) transduces chimpanzee DCs very poorly in comparison to Ad5.Fib35 (66.5%).

### Example VI

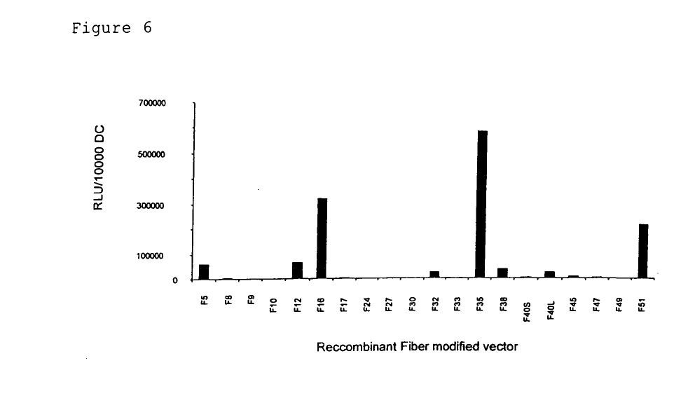

Immature DCs were incubated with Ad5.Luc or with the fiber-modified vectors at a virus dose of 10⁵ virus particles per DC. Luciferase transgene expression was determined 24 hours after virus exposure. Results are a representative of two independent experiments performed with DC derived from 2 different individuals, and depicted graphically in FIG. 6. Results are expressed in relative light units (“RLU”) per 10⁴ DC versus recombinant fiber modified vector.

### Example VII

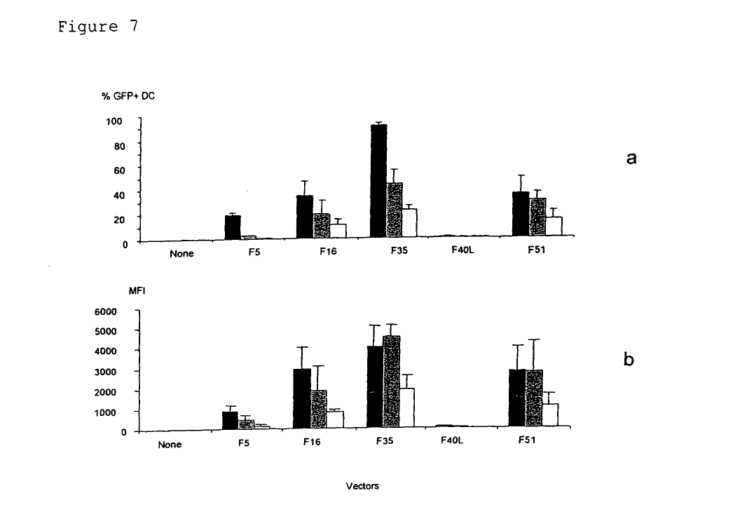

Following-up on Example V, immature DCs were incubated with Ad5.GFP or Ad5Fib16.GFP, Ad5Fib35.GFP, Ad5fib40-L.GFP, or Ad4.Fib51.GFP. Different dosages were used: 10³, 10⁴, or 10⁵ virus particles per DC (white bar, grey bar, or black bar respectively in FIG. 7). GFP expression was determined 24 hours after virus exposure. FIG. 7 expresses the results in Graph a as percentage GFP positive cells and, in Graph b, as median fluorescence intensity. Results shown are derived from 3 independent experiments performed with DC derived from 3 different individuals.

### Example VIII

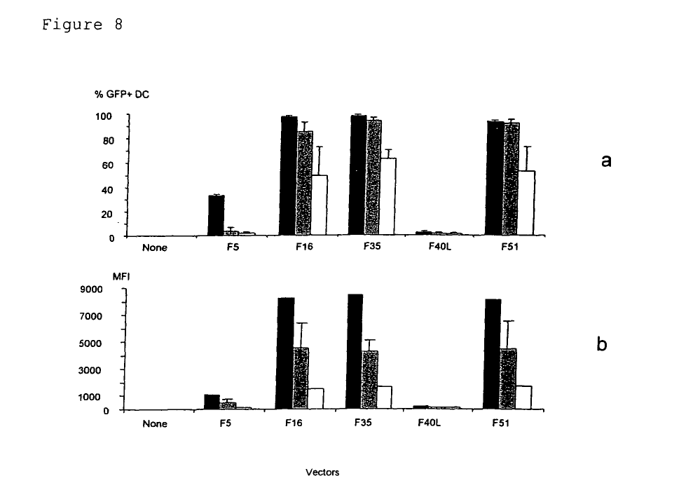

Immature DC were treated for 48 hours with LPS to allow maturation of the DC. Matured DCs were incubated with Ad5.GFP or Ad5Fib16.GFP, Ad5Fib35.GFP, Ad5fib40-L.GFP, or Ad4.Fib51.GFP. Dosages used were 10³, 10⁴, or 10⁵ virus particles per DC (depicted as white, grey, and black bars respectively in FIG. 8). GFP expression was determined 24 hours after virus exposure. In FIG. 8, results are expressed as (a) percentage GFP positive cells and (b) median fluorescence intensity (b). Results shown are derived from 3 independent experiments performed with DC derived from 3 different individuals.

### Example IX

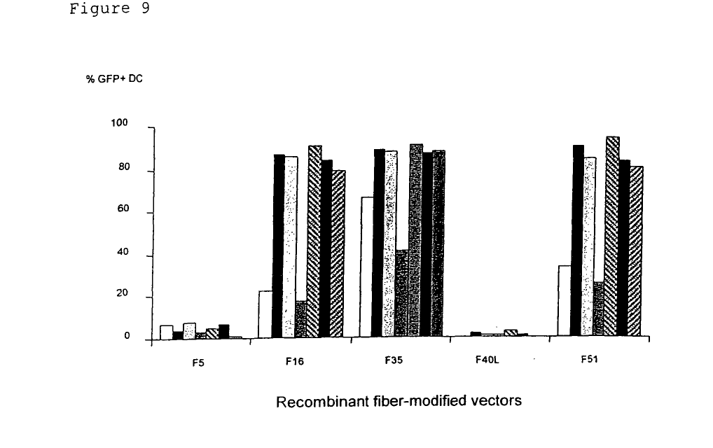

Immature dendritic cells were exposed for 48 hours to different maturation agents before being exposed to various viruses. The virus dosage used was 10⁴ virus particles per DC. GFP expression was determined 24 hours after virus exposure. The results are expressed as percentage GFP positive cells. Results shown are representative of 2 independent experiments. The results are graphically depicted in FIG. 9. The maturation agents used were LPS (black bars), TNF-a (white with black dots), MCM (diagonal downward), poly I:C (black with white dots), and anti-CD40 antibodies (diagonal upwards). As a negative control for a maturation marker, IFN-a (grey bars) was used, while immature dendritic cells were used as a general control (white bars).

### Example X

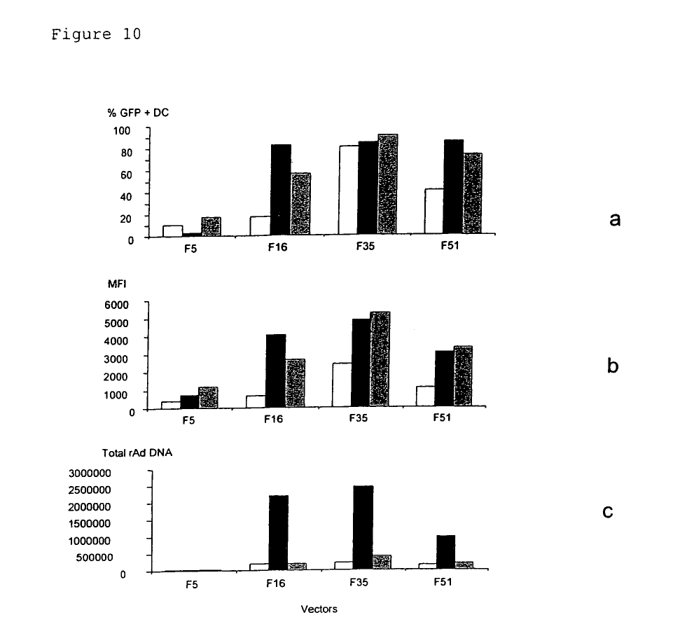

Dendritic cells were exposed to 10⁴ virus particles per cell where various vectors (F5, F16, F35, and F51) were used. The results are graphically depicted in FIG. 10, wherein, in (a), the percentage GFP positive cells detected is depicted, in (b) the median fluorescence intensity is depicted and, in (c), cells that were frozen and genomic DNA extracted to quantify the number of adenoviral genomes using real-time PCR. The DC types were immature DC (white bar), mature DC (black bar), or immature DC transduced and subsequently matured using LPS. Cells were analyzed for GFP expression 48 hours after virus exposure. The data are representative for two independent experiments.

### Example XI

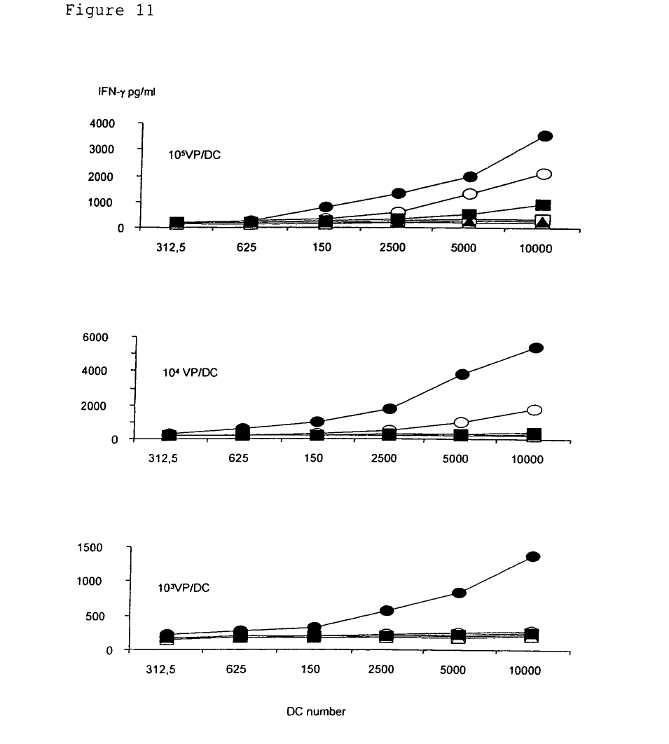

As graphically depicted in FIG. 11, immature dendritic cells were transduced with 10⁵, 10⁴ or 10³ virus particles (top, middle, and bottom graphs respectively) of Ad5.gp100 or Ad5.Fib35.gp100 (white squares and circles, respectively). Likewise, matured DC were transduced with 10⁵, 10⁴ or 10³ virus particles of Ad5.gp100 or Ad5.Fib35.gp100 (black squares and circles respectively). Transduced DC (10⁴ cells) were cultured with the HLA-A2-restricted 8J CTL clone (10⁴ cells) and IFN-gamma production which demonstrates CTL activation, which was determined 24 hours later.

### Example XII

Immature DC and mature DC (LPS) were exposed to 10⁴ virus particles per dendritic cell. Forty-eight hours after virus exposure, the cells were analyzed for expression of membrane proteins CD86, CD83, HLA-class I, and HLA-DR. Also, release of IL-12 was measured. As a control, non-transduced immature DC and mature DC were used. Results for the membrane proteins are expressed in mean fluorescence intensity (Table 1). Results for IL-12 production are expressed in pg/ml.

TABLE 1
Effect of fiber-modified adenoviral vectors on the ability of DC to
maturate.

| | | | | | IL-12 p70 |
|---|---|---|---|---|---|
| DC type | CD86* | HLA-DR* | HLA-class I* | CD83* | (pg/ml) |
| IDC | 99 | 724 | 553 | 8 | 0 |
| LPS | 1786 | 1697 | 1906 | 96 | 324 |
| F5 | 232 | 889 | 627 | 17 | 0 |
| F5 LPS | 2020 | 2392 | 2439 | 107 | 3647 |
| F16 | 460 | 1038 | 769 | 19 | |
| F16 LPS | 2474 | 2152 | 2694 | 143 | 5628 |
| F35 | 751 | 1278 | 750 | 34 | 0 |
| F35 LPS | 2298 | 2380 | 2360 | 157 | 12944 |
| F51 | 446 | 1086 | 638 | 19 | 0 |
| F51 LPS | 2633 | 2502 | 2351 | 132 | 9267 |

Although the present invention has been described with respect to the illustrated embodiments, various additions, deletions and modifications are contemplated as being within its scope. The scope of the invention is, therefore, indicated by the ensuing claims, rather than the foregoing description. All changes that come within the meaning and range of equivalency of the claims are to be embraced within their scope.

### REFERENCES

Amin et al. “Nitric Oxide Synthase and Cyclooxygenase: Distribution, Regulation and Intervention in Arthritis” Curr Opin Rheumatol. 11, pp. 202-209 (1999).

Arnberg et al. “Fiber Genes of Adenoviruses with Tropism for the Eye and the Genital Tract”, Virology 227:239-244 (1997).

Bergelson, et al. “Isolation of a Common Receptor for Coxsackie B Virus and Adenoviruses 2 and 5”, Science 275:1320-1323 (1997).

A. Bout “Gene Therapy”, pp. 167-182, in: D. J. A. Crommelin and R. D. Sindelar (editors), Pharmaceutical Biotechnology, Harwood Academic Publishers (1997).

A. Bout “Prospects for Human Gene Therapy” Eur. J Drug Met. and Pharma, 2:175-179(1996).

Blaese et al. Cancer Gene Ther. 2:291-297 (1995).

Brody, S. L. and Crystal, R. G. “Adenovirus Mediated in Vivo Gene Transfer” Ann. N.Y. Acad. Sci. 716:90-101 (1994).

Chroboczek et al. “Adenovirus fiber”, pp. 163-200 in: W. Doerfler and P. Böhm (editors), The Molecular Repertoire of Adenoviruses, I. (Springer-Verlag, Berlin, 1995).

Defer et al. “Human Adenovirus-host Cell Interactions: Comparative Study with Members of Subgroup B and C” Journal of Virology, 64 (8):3661-3673 (1990).

Fallaux et al. “New Helper Cells and Matched El-deleted Adenovirus Vectors Prevent Generation of Replication Competent Adenoviruses” Human Gene Therapy, 9, pp.1909-1917(1998).

Fernandez et al. “In Vivo Transfer of Interleukin-1 Receptor Antagonist Gene in Osteoarthritic Rabbit Knee Joints: Prevention of Osteoarthritis Progression”, Am J Pathol, 154, pp. 1159-1169(1999).

Francki et al. “Classification and Nomenclature of Viruses. Fifth Report of the International Committee on Taxonomy of Viruses”, Arch. Virol. Suppl., 2:140-144 (1991).

Frenkel, S R and DiCesare, P E “Degradation and Repair of Articular Cartilage”, Front Biosci 15(4):671-685 (1999).

Gazit et al. “Engineered Pluripotent Mesenchymal Cells Integrate and Differentiate in Regenerating Bone: a Novel Cell-mediated Gene Therapy”, J Gene Med 1, pp.121-133 (1999).

Gall et al. “Adenovirus Type 5 and 7 Capsid Chimera: Fiber Replacement Alters Receptor Tropism Without Affecting Primary Immune Neutralization Epitopes”, Journal of Virology 70(4):2116-23 (1996).

Goto et al. “Transfer of LacZ marker gene to the meniscus”, J Bone Joint Surg Am, 81:918-925 (1999).

Greber et al. “Stepwise Dismantling of Adenovirus 2 During Entry into Cells”, Cell 75:477-486 (1993).

R. O. Hynes “Integrins: Versatility, Modulation and Signaling in Cell Adhesion”, Cell 69:11-25 (1992).

Herz, J. and Gerard, R. D. “Adenovirus-mediated Transfer of Low Density Lipoprotein Receptor Gene Acutely Accelerates Cholesterol Clearance in Normal Mice”, Proc. Natl. Acad. Sci. U.S.A., 962812-16 (1993).

J. C. Hierholzer, “Adenovirus in the Immunocompromised Host”, Clin. Microbiol Rev., 5:262-74 (1992).

Hierholzer et al. “Adenoviruses from Patients with AIDS: A Plethora of Serotypes and a Description of Five New Serotypes of Subgenus D (Types 43-47)”, J. Infect. Dis., 158:804-813 (1988).

Hong et al. “Adenovirus Type 5 Fiber Knob Binds to MHC Class I (2 Domain at the Surface of Human Epithelial and B Lymphoblastoid Cells”, EMBO J. 16:2294-2306 (1997).

Hsu et al. “A Monoclonal Antibody Specific for the Cellular Receptor for the Group B Coxsackieviruses”, J. Virol 62(5): 1647-52 (1988).

Huard et al. “The Route of Administration Is a Major Determinant of the Transduction Efficiency of Rat Tissues by Adenoviral Recombinants” Gene Ther. 2: 107-115 (1995).

Ishibashi, M. and Yasue, H. “The Adenoviruses,” H. S. Ginsberg, ed., Chapter 12, 497-561 (Plenum Press, London, New York. 1984).

Kass-Eisler et al. “The Impact of Developmental Stage, Route of Administration and the Immune System on Adenovirus-mediated Gene Transfer”, Gene Ther., 1:395-402 (1994).

Khoo et al. “Adenovirus Infections in Human Immunodeficiency Virus-positive Patients: Clinical Features and Molecular Epidemiology”, J. Infect. Dis 172, 629-637 (1995).

Kidd et al. “Adenovirus Type 40 Virions Contain Two Distinct Fibers”, Virology, 192:73-84 (1993).

Kramer et al, “Embryonic Stem Cell-derived Chondrogenic Differentiation in V Activation by BMP-2 and BMP-4” Mech Dev 92:193-205.

Krasnykh et al. “Generation of Recombinant Adenovirus Vectors with Modified Fibers for Altering Viral Tropism”, J. Virol, 70(10):6839-6846 (1996).

Krasnykh et al. “Characterization of an Adenovirus Vector Containing a Heterologous Peptide Epitope in the Hi Loop of the Fiber Knob” J. Virol. 72(3):1844-1852 (1998).

Law et al. “Infection of Primary CNS Cells by Different Adenoviral Serotypes: Searching for a More Efficient Vector”, Abstract 1St Annual Meeting American Society of Gene Therapy, Seattle, Wash. (1998).

K. N. Leppard “E4 Gene Function in Adenovirus, Adenovirus Vector and Adeno-associated Virus Infections”, J. Gen. Virol., 78:2131-2138 (1997).

Lubberts et al. “Adenoviral Vector-mediated Gene Delivery to the Temporomandibular Joint in Guinea-pigs”, Arch. Oral Biol, 44:701-709 (1999).

Madry, H and Trippel, SB “Efficient Lipid-mediated Gene Transfer to Articular Chondrocytes” Gene Ther, 7:286-291 (2000).

Mason et al. “Expression of Human Bone Morphogenesis Protein 7 in Primary Rabbit Periosteal Cells: Potential Utility in Gene Therapy for Osteochondral Repair”, Gene Ther 5:1098-1104 (199_.

Morgan et al. “Structure and Development of Viruses as Observed in the Electron Microscope.x. Entry and Uncoating of Adenovirus”, J. Virol 4, 777-796 (1969).

Nishida et al. “Modulation of the Biological Activity of the Rabbit Intervertebral Disc by Gene Therapy: an in Vivo Study of Adenovirus-mediated Transfer of the Human Transforming Growth Factor Beta 1 Encoding Gene”, Spine 24:2419-2425 (1999).

Pizette, S and Niswander, L “BMPs are Required at Two Steps of Limb Chondrogenesis: Formation of Prechondrogenic Condensations and Their Differentiation into Chondrocytes”, Dev Biol 219:237-249 (2000).

Richardson et al. “Repair of human cartilage after implantation of autologous chondrocytes”, J Bone Joint Surg. Br 81:1064-1068 (1999).

Roelvink et al. “Comparative Analysis of Adenovirus Fiber-cell Interaction: Adenovirus Type 2 (Ad2) and Ad9 Utilize the Same Cellular Fiber Receptor but Use Different Binding Strategies for Attachment”, J. Virol., 70:7614-7621 (1996).

Roelvink et al. “The Coxsackie-adenovirus Receptor Protein Can Function as a Cellular Attachment Protein for Adenovirus Serotypes from Subgroups A, C, D, E, and F”, J. Virol. 72:7909-7915 (1998).

Rogers et al. “Use of a Novel Cross-linking Method to Modify Adenovirus Tropism”, Gene Ther. 4:1387-1392 (1997).

Risau, W. “Mechanisms of Angiogenesis”, Nature, 386:671-674 (1997).

Schulick, A. H., Vassalli, G., Dunn, P. F., Dong, G., Rade, J. J., Zamarron, C. and Dichek, D. A. “Established Immunity Precludes Adenovirus-mediated Gene Transfer in Rat Carotid Arteries”, (1997).

Schnurr, D and Dondero, M. E. “Two new candidate adenovirus serotypes”, Intervirol., 36:79-83 (1993).

Shabram et al. Analytical Anion-Exchange HPLC of Recombinant Type-5 Adenoviral Particles”, Hum. Gene Ther., 8(4):453-465 (1997).

Silverman et al. “Adhesion of Tissue-engineered Cartilage to Native Cartilage”, Plast Reconstr. Surg 105:1393-1398 (2000).

Signas et al. “Adenovirus 3 Fiber Polypeptide Gene: Complications for the Structure of the Fiber Protein”, J. Virol. 53:672-678 (1985).

Stevenson et al. “Human Adenovirus Serotypes 3 and 5 Bind to Two Different Cellular Receptors via the Fiber Head Domain”, J. Virol. 69(5):2850-2857 (1995).

Stevenson et al. “Selective Targeting of Human Cells by a Chimaeric Adenovirus Vector Containing a Modified Fiber Protein”, J. Virology 71(6):4782-4790 (1997).

Stouten et al. “New Triple Helical Model for the Shaft of the Adenovirus Fiber”, J. Mol. Biol., 226:1073-1084 (1992).

Svensson, V. and Persson, R. “Entry of Adenovirus 2 into Hela cells”, J. Virol. 51:687-694 (1984).

Taniguchi et al. “Induction of the p16INK4a Senescence Gene as a New Therapeutic Strategy for the Treatment of Rheumatoid Arthritis”, Nat Med 5:760-767 (1999).

P. C. van der Vliet “Adenovirus DNA Replication” in: W. Doerfler and P. Böhm (editors), The Molecular Repertoire of Adenoviruses II. (Springer-Verlag, Berlin 1995).

Varga et al. “Infectious Entry Pathway of Adenovirus Type 2”, J. Virol, 65:6061-6070 (1991).

G. Wadell “Molecular Epidemiology of Human Adenoviruses”, Curr. Top. Microbiol. Immunol. 110:191-220 (1984).

Whalen et al. “Adenoviral Transfer of the Viral IL-10 Gene Periarticularly to Mouse Paws Suppresses Development of Collagen-induced Arthritis in Both Injected and Uninjected Paws”, J Immunol, 162:3625-3632 (1999).

Wickham et al. “Targeting of Adenovirus Penton Base to New Receptors Through Replacement of its RGD Motif with Other Receptor-specific Peptide Motifs”, Gene Therapy 2:750-756 (1995).

Wickham et al. “Integrins avb3 and avb5 Promote Adenovirus Internalization but Not Virus Attachment”, Cell 73, 309-319 (1993).

Wold et al. “E3 Transcription Unit of Adenovirus” in: W. Doerfler and P. Böhm (editors), The Molecular Repertoire of Adenoviruses I, (Springer-Verlag, Berlin 1995).

Zabner et al. “Type 17 Fiber Enhances Gene Transfer”, Abstract 1^{st} Annual Meeting American Society of Gene Therapy, Seattle, Wash. (1998).

What is claimed is:

1. A method for delivering a heterologous nucleic acid to a dendritic cell, said method comprising:
providing a recombinant adenoviral vector that includes a tropism for dendritic cells and comprises the heterologous nucleic acid; and
exposing the dendritic cell to the recombinant adenoviral vector,
thus delivering said heterologous nucleic acid to the dendritic cell.

2. The method according to claim 1, wherein providing the recombinant adenoviral vector comprises providing a recombinant adenoviral vector with an at least partially reduced tropism for liver cells.

3. The method according to claim 1, wherein providing the recombinant adenoviral vector comprises providing a recombinant adenoviral vector including at least a part of an adenoviral capsid protein having a tropism for dendritic cells.

4. The method according to claim 1, wherein providing the recombinant adenoviral vector comprises providing a recombinant adenoviral vector including adenoviral capsid proteins from at least two different adenovirus serotypes.

5. The method according to claim 4, wherein at least one of the at least two different adenovirus serotypes is selected from adenovirus subgroup B.

6. The method according to claim 5, wherein at least one of the at least two different adenovirus serotypes is selected from adenovirus subgroup C.

7. The method according to claim 4, wherein at least one of the at least two different adenovirus serotypes is selected from the group consisting of adenovirus serotypes 11, 16, 35, 51, and 40L.

8. The method according to claim 1, wherein providing the recombinant adenoviral vector comprises providing a recombinant adenoviral vector based on a first adenovirus of adenovirus subgroup C with at least a part of a non-native fiber protein substituted for at least a part of a native fiber protein of the first adenovirus, the part of a non-native fiber protein selected from the group consisting of fiber proteins from adenovirus serotypes 11, 16, 35, 51, and 40L.

9. The method according to claim 1, wherein providing the recombinant adenoviral vector comprises providing a recombinant adenoviral vector based on a first adenovirus of adenovirus subgroup C with at least a part of a fiber protein from adenovirus serotype 35 substituted for at least a part of a native fiber protein of the first adenovirus.

10. The method according to claim 1, wherein providing the recombinant adenoviral vector comprises providing a recombinant adenoviral vector based on a first adenovirus of adenovirus subgroup C with at least a part of a fiber protein from adenovirus serotype 16 substituted for at least a part of a native fiber protein of the first adenovirus.

11. The method according to claim 1, wherein providing the recombinant adenoviral vector comprises providing a recombinant adenoviral vector based on a first adenovirus of adenovirus subgroup C with at least a part of a fiber protein from adenovirus serotype 11 substituted for at least a part of a native fiber protein of the first adenovirus.

12. The method according to claim 1, wherein providing the recombinant adenoviral vector comprises providing a recombinant adenoviral vector based on a first adenovirus of adenovirus subgroup C with at least a part of a fiber protein from adenovirus serotype 51 substituted for at least a part of a native fiber protein of the first adenovirus.

13. The method according to claim 1, wherein providing the recombinant adenoviral vector comprises providing a recombinant adenoviral vector based on a first adenovirus of adenovirus subgroup C with at least a part of a fiber protein from adenovirus serotype 40L substituted for at least a part of a native fiber protein of the first adenovirus.

14. The method according to claim 1, wherein providing the recombinant adenoviral vector comprises providing a recombinant adenoviral vector based on a first adenovirus of serotype 5 with at least a part of a non-native fiber protein substituted for at least a part of a native fiber protein of the first adenovirus, the part of a non-native fiber protein selected from the group consisting of fiber proteins from adenovirus serotypes 11, 16, 35, 51, and 40L.

15. The method according to claim 1, wherein providing the recombinant adenoviral vector comprises providing a recombinant adenoviral vector based on a first adenovirus of serotype 5 with at least a part of a fiber protein from adenovirus serotype 35 substituted for at least a part of a native fiber protein of the first adenovirus.

16. The method according to claim 1, wherein providing the recombinant adenoviral vector comprises providing a recombinant adenoviral vector based on a first adenovirus of serotype 5 with at least a part of a fiber protein from adenovirus serotype 16 substituted for at least a part of a native fiber protein of the first adenovirus.

17. The method according to claim 1, wherein providing the recombinant adenoviral vector comprises providing a recombinant adenoviral vector based on a first adenovirus of serotype 5 with at least a part of a fiber protein from adenovirus serotype 11 substituted for at least a part of a native fiber protein of the first adenovirus.

18. The method according to claim 1, wherein providing the recombinant adenoviral vector comprises providing a recombinant adenoviral vector based on a first adenovirus of serotype 5 with at least a part of a fiber protein from adenovirus serotype 51 substituted for at least a part of a native fiber protein of the first adenovirus.

19. The method according to claim 1, wherein providing the recombinant adenoviral vector comprises providing a recombinant adenoviral vector based on a first adenovirus of serotype 5 with at least a part of a fiber protein from adenovirus serotype 40L substituted for at least a part of a native fiber protein of the first adenovirus.

20. The method according to claim 1, wherein providing the recombinant adenoviral vector comprises providing a recombinant adenoviral vector modified such that replication of the recombinant adenoviral vector's genome in a target cell is at least partly reduced in comparison to a wild-type adenovirus.

21. The method according to claim 1, wherein providing the recombinant adenoviral vector comprises providing a recombinant adenoviral vector modified such that an immune response to the recombinant adenoviral vector in a host is at least partly reduced in comparison to a wild-type adenovirus.
